(19) United States
(12) Patent Application Publication
(10) Pub. No.: US 2025/0136602 A1
(43) Pub. Date: May 1, 2025

(54) PROCESS FOR THE PREPARATION OF A PURE AMORPHOUS FORM OF UBROGEPANT

(71) Applicant: MSN LABORATORIES PRIVATE LIMITED, F&D CENTER, Hyderabad, Telangana (IN)

(72) Inventors: Thirumalai Rajan SRINIVASAN, Hyderabad, Telangana (IN); Eswaraiah SAJJA, Hyderanad, Telangana (IN); Rajeshwar Reddy SAGYAM, Hyderabad, Telangana (IN); Parameshwarappa GANGAJJI, Hyderabad, Telangana (IN); Praveen VELISHALA, Hyderabad, Telangana (IN)

(73) Assignee: MSN LABORATORIES PRIVATE LIMITED, F&D CENTER, Hyderabad, Telangana (IN)

(21) Appl. No.: 18/722,385

(22) PCT Filed: Dec. 20, 2022
(86) PCT No.: PCT/IN2022/051108

§ 371 Date: Jun. 20, 2024

(30) Foreign Application Priority Data
Dec. 20, 2021 (IN) 202141059433

(51) Int. Cl. C07D 471/20

(52) U.S. Cl. CPC C07D 471/20

(57) ABSTRACT

The present application relates to a pure amorphous form of Ubrogepant and its process for the preparation thereof. Ubrogepant is chemically known as (3′S)—N-((3S, 5S, 6R)-6-methyl-2-oxo-5-phenyl-1-(2, 2, 2trifluoroethyl) piperidin-3-yl)-2′-oxo-1′, 2′, 5, 7-tetrahydro spiro [cyclopenta [b] pyridine-6, 3′-pyrrolo [2, 3b] pyridine]-3-carboxamide and it is represented by the following structural formula-1.

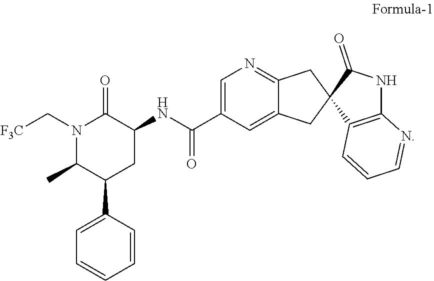 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F]

This application claims the benefit of priority of our Indian Patent Application number 202141059433 filed on Dec. 20, 2021, which is incorporated herein by reference.

### FIELD OF THE INVENTION

The present application relates to pure amorphous form of Ubrogepant and its process for the preparation thereof. Ubrogepant represented by the following structural formula-1.

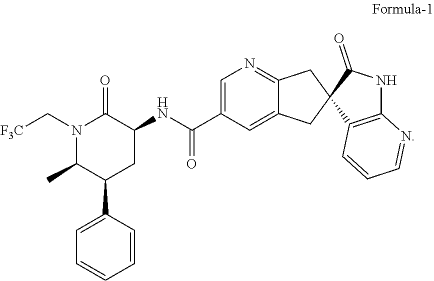 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F]

### BACKGROUND OF THE INVENTION

Ubrogepant formula-1 is chemically known as (3′S)—N-((3S,5S,6R)-6-methyl-2-oxo-5-phenyl-1-(2,2,2-trifluoroethyl) piperidin-3-yl)-2′-oxo-1′,2′,5,7-tetrahydrospiro [cyclopenta[b]pyridine-6,3′-pyrrolo[2,3-b]pyridine]-3-carboxamide, which was approved for the indication of calcitonin gene-related peptide receptor antagonist indicated for the acute treatment of migraine with or without aura in adults with the brand name of Ubrelvy®.

Ubrogepant or its pharmaceutically acceptable salts described in U.S. Pat. No. 8,754,096B2. This also discloses the process for the preparation of Ubrogepant.

U.S. Pat. No. 9,174,989B2 patent describes the various polymorphic forms of Ubrogepant such as Ubrogepant monohydrate, Ubrogepant trihydrate, Ubrogepant acetonitrile solvate, Ubrogepant acetonitrile water solvate, Ubrogepant pseudo amorphous and Ubrogapant L-tartaric acid co-crystal.

U.S. Pat. No. 9,174,989B2 describes the preparation of pseudo-amorphous form of Ubrogepant by drying of Ubrogepant acetonitrile-water solvate at 75° C. under vacuum for one hour, which is generated via desolvation of an acetonitrile solvate. The X-ray amorphous pattern displays a broad diffuse halo with only a single low angle peak at approximately 5° two-theta.

The methods for preparation of amorphous Ubrogepant described in U.S. Pat. No. 9,174,989B2 reference suffer from many shortcomings, such as need to prepare Ubrogepant acetonitrile-water solvate, further obtained Ubrogepant is in pseudo-amorphous form. The prior art fails to describe the purity and level of the residual solvents in final product.

Discovering new solid-state forms/polymorphic forms, solvates of a pharmaceutical product can provide materials having desirable physicochemical properties. New polymorphic forms and solvates of a pharmaceutically useful compound can also provide an opportunity to improve the performance characteristics of a pharmaceutical product.

Crystalline solids normally require a significant amount of energy for dissolution due to their highly organized, lattice like structures. For example, the energy required for a drug molecule to escape from a crystal is more than from an amorphous or a non-crystalline form. It is known that the amorphous forms in a number of drugs exhibit different dissolution characteristics and in some cases different bioavailability patterns compared to the crystalline form (Econno T., Chem. Pharm. Bull., 1990; 38:2003-2007). For some therapeutic indications, one bioavailability pattern may be favored over another.

An amorphous form of some of the drugs exhibit much higher bioavailability than the crystalline forms, which leads to the selection of the amorphous form as the final drug substance for pharmaceutical dosage from development. Additionally, the aqueous solubility of crystalline form is lower than its amorphous form in some of the drugs, which may have resulted in the difference in their in-vivo bioavailability. Therefore, it is desirable to have amorphous forms of drugs with high purity to meet the needs of regulatory agencies and also highly reproducible processes for their preparation.

As discussed herein above, the prior art processes provides a pseudo-amorphous Ubrogepant with low purity and high residual solvents which is not suitable for pharmaceutical preparations. Therefore, there still exists a need to provide an efficient process for preparation of pure amorphous Ubrogepant with higher purity and lower levels of residual solvent(s) in accordance with the pharmaceutical requirements, which is suitable for pharmaceutical preparations.

### SUMMARY OF THE INVENTION

First embodiment of the present invention provides a pure amorphous form of Ubrogepant of formula-1.

Second embodiment of the present provides a process for the preparation of pure amorphous form of Ubrogepant of formula-1.

### BRIEF DESCRIPTION OF THE DRAWINGS

FIG. 1: Illustrates the powder X-Ray diffraction {PXRD} pattern of pure amorphous form of Ubrogepant.

FIG. 2: Illustrates the powder X-Ray diffraction {PXRD} pattern of pure amorphous form of Ubrogepant.

FIG. 3: Illustrates the powder X-Ray diffraction {PXRD} pattern of pure amorphous form of Ubrogepant.

### DETAILED DESCRIPTION OF THE INVENTION

The “suitable solvent” used in the present invention can be selected from but not limited to “hydrocarbon solvents” such as n-pentane, n-hexane, n-heptane, cyclohexane, petroleum ether, benzene, toluene, xylene and mixtures thereof; “ether solvents” such as dimethyl ether, diethyl ether, diisopropyl ether, methyl tert-butyl ether, 1,2-dimethoxyethane, tetrahydrofuran, 1,4-dioxane and mixtures thereof; “ester solvents” such as methyl acetate, ethyl acetate, n-propyl acetate, isopropyl acetate, n-butyl acetate, isobutyl acetate, tert-butyl acetate and mixtures thereof; “polar-aprotic solvents” such as dimethylacetamide, dimethylformamide, dimethylsulfoxide, N-methylpyrrolidone (NMP) and mixtures thereof; “chloro solvents” such as dichloromethane, dichloroethane, chloroform, carbon tetrachloride and mixtures thereof; “ketone solvents” such as acetone, methyl ethyl ketone, methyl isobutyl ketone and mixtures thereof; “nitrile solvents” such as acetonitrile, propionitrile, isobutyronitrile and mixtures thereof; “alcohol solvents” such as methanol, ethanol, n-propanol, iso-propanol, n-butanol, iso-butanol, 2-butanol, tert-butanol, ethane-1,2-diol, propane-1,2-diol and mixtures thereof; “polar solvents” such as water; formic acid, acetic acid and the like or mixture of any of the afore mentioned solvents.

All ranges recited herein include the endpoints and the terms “about”, “from”, “to” are to be construed as modifying a value they are applied to such that it is not absolute and includes, to the very least, the degree of expected experimental error, limitation of method or instrument error for a given technique used to measure the value.

As used herein, the term “solution” or “reaction mixture” does not limit to a clear solution only and includes any hazy or opaque mass obtained.

The first embodiment of the present invention provides a pure amorphous form of Ubrogepant of formula-1.

In the first aspect of the first embodiment, pure amorphous form of Ubrogepant having purity of greater than about 99% or greater than about 99.5% or greater than about 99.7% measured by HPLC {High Performance Liquid Chromatography}.

In the second aspect of the first embodiment, pure amorphous form of Ubrogepant is essentially free of residual solvents. “Essentially free of residual solvents” herein refers to pure amorphous form of Ubrogepant having lower levels of residual solvent(s) in accordance with the pharmaceutical requirements, which is suitable for pharmaceutical preparations.

In the third aspect of the first embodiment, pure amorphous form of Ubrogepant contains less than about 5% or less than about 3% or less than about 2% or less than about 1% or less than about 0.5% or less than about 0.3% or less than about 0.2% or less than about 0.1% of crystalline forms of Ubrogepant.

In the fourth aspect of the first embodiment, pure amorphous form of Ubrogepant is essentially free of crystalline forms of Ubrogepant. “Essentially free of crystalline forms of Ubrogepant” means that no crystalline polymorph forms of Ubrogepant can be detected within the limits of a given powder X-ray diffraction method.

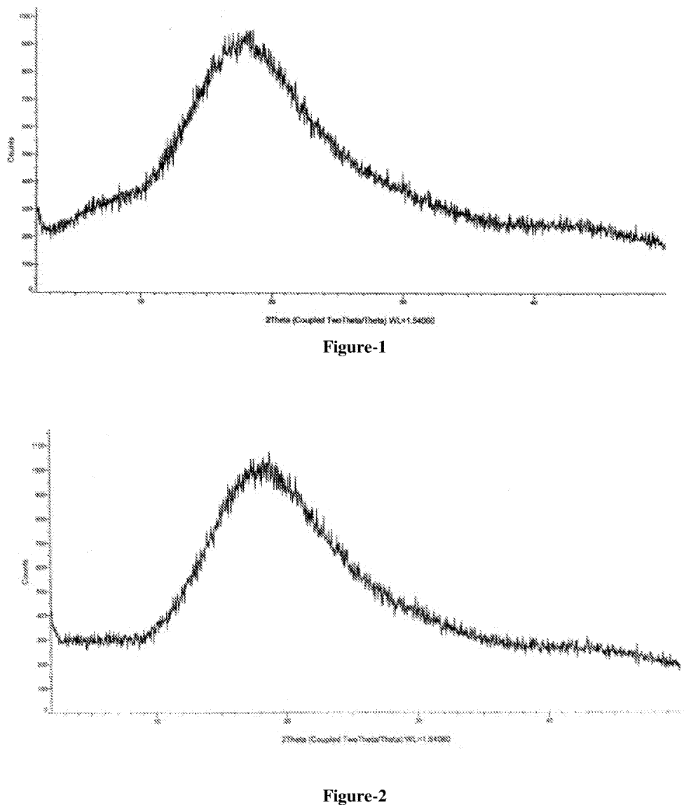

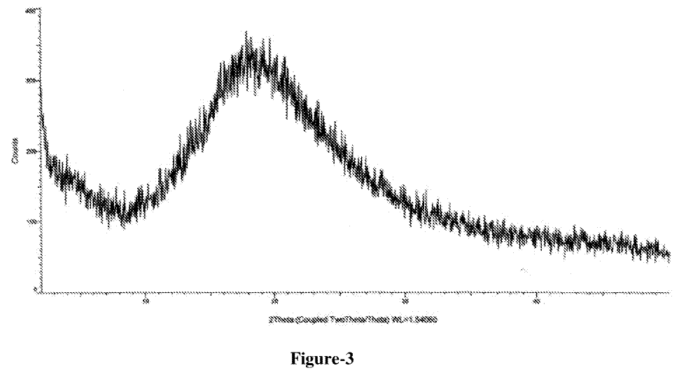

In fifth aspect of the first embodiment, pure amorphous form of Ubrogepant is characterized by a powder X-ray diffraction (PXRD) pattern is illustrated by FIG. 1 or FIG. 2 or FIG. 3.

Sixth aspect of first embodiment, pure amorphous form of Ubrogepant is stable which is suitable for pharmaceutical preparations with having greater stability.

The term “stable” includes amorphous Ubrogepant that does not convert to any other solid form when stored at a temperature of up to about 40° C. and at a relative humidity of about 25% to about 75% for about six months or more.

Seventh aspect of the first embodiment, there is provided a stable amorphous Ubrogepant that does not convert to any other solid forms when stored at a temperature of up to about 40° C. and at a relative humidity of about 25% to about 75% for about three months or more.

Eighth aspect of the first embodiment, the accelerated and long-term stability data of amorphous Ubrogepant of formula-1 is outlined in Table-1 as follows:

| | Purity by HPLC | PXRD Result |
|---|---|---|
| Long term stability at 25 ± 2° C. | >99.75% | Amorphous |
| under 60 ± 5% humidity | | Ubrogepant is |
| condition upto 6 months | | stable |
| Accelerated stability at 40 ± 2° C. | >99.75% | Amorphous |
| under 75 ± 5% humidity | | Ubrogepant is |
| condition upto 6 months | | stable |

Ninth aspect of the first embodiment of the present invention, amorphous Ubrogepant is also stable under below Polymorphic stress study conditions:

| Stress Condition | Duration | | Result |
|---|---|---|---|
| Stress Study under 10 tons | 3 | mins | Amorphous Ubrogepant is |
| pressure | | | stable |
| Thermal Analysis at 60° ± 2° C. | 24 | hours | Amorphous Ubrogepant is |
| | | | stable |
| Under UV light at 254 nm | 24 | hours | Amorphous Ubrogepant is |
| | | | stable |
| Hygroscopicity analysis | 24 | hours | Amorphous Ubrogepant is |
| (75 ± 5% RH) | | | stable |

The second embodiment of the present invention provides a process for the preparation of amorphous form of Ubrogepant of formula-1, the process comprising the steps of:
a) dissolving Ubrogepant of formula-1 in a solvent,
b) isolating pure amorphous form of Ubrogepant.
   Wherein, dissolving Ubrogepant in step-a) can be carried out at a temperature ranging from about 25° C. to reflux temperature of the solvent used. The solvent in step-a) is selected from ester solvent, alcohol solvent, ketone solvent, polar-aprotic solvents; isolating pure amorphous form of Ubrogepant in step-b) by known techniques which are selected from removing of the solvent, combining with an anti-solvent, distillation, decanting, filtration, cooling the mixture to lower temperatures to precipitate the solid followed by filtration of the mixture. The anti-solvent is a selected from ether solvents, hydrocarbon solvents water or mixture thereof.

An aspect of second embodiment of the present invention provides a process comprising the steps of:
a) dissolving Ubrogepant of formula-1 in a solvent,
b) isolating amorphous form of Ubrogepant combining with an anti-solvent.
   Wherein, dissolving Ubrogepant in step-a) can be carried out at a temperature ranging from about 25° C. to reflux temperature of the solvent used. The solvent in step-a) is selected from ester solvent, alcohol solvent, ketone solvent, nitrile solvent, polar-aprotic solvents or mixtures thereof; anti-solvent in step-b) is selected from ether solvents, hydrocarbon solvents water or mixture thereof.

In general, the amorphous Ubrogepant obtained according to the present invention has particle size distribution as characterized by 90% particles having particle size (D90) less than about 100 μm, 50% particles having particle size (D50) less than about 50 μm and 10% particles having particle size (D10) less than about 10 μm.

Amorphous form of Ubrogepant produced by the processes of the present invention can be further micronized or milled to get desired particle size to achieve desired solubility profile based on different forms of pharmaceutical composition requirements. Techniques that may be used for particle size reduction include but not limited to single or multi-stage micronization using cutting mills, pin/cage mills, hammer mills, jet mills, fluidized bed jet mills, ball mills and roller mills. Milling or micronization may be performed before drying or after drying of the product.

### HPLC Method of Analysis:

Ubrogepant and its related substances were analyzed by HPLC with the following chromatographic conditions:
Apparatus: A liquid chromatographic system is to be equipped with variable wavelength UV-Detector and integrator; Column: Xbridge C18 150×4.6 mm, 3.5 μm
Ghost-Buster column: Ghost buster 50 mm×4.6 mm; Wavelength: 210 nm; Column temperature: 25° C.; Injection volume: 5 μL; Elution: Gradient; Diluent: Acetonitrile: Water; Needle wash: Diluent.

### Buffer Preparation:

(i) Accurately transfer 1000 ml of milli-Q-water into a suitable cleaned and dry beaker.
(ii) Transfer accurately 1 ml of Ortho phosphoric acid (H₃PO₄) to above 1000 ml of milli-Q-water, sonicate to dissolve and mix well.
(iii) Filter the above solution through 0.22 μm PVDF filter paper and sonicate to degas it.
   Mobile phase-A: Buffer (100%); Mobile phase-B: Acetonitrile: Water

An embodiment of the present invention provides the use of pure amorphous form of Ubrogepant of formula-1 of the present invention for the preparation of various pharmaceutical formulations.

Another embodiment of the present invention provides pharmaceutical composition comprising pure amorphous form of Ubrogepant of formula-1 obtained according to the present invention and at least one pharmaceutically acceptable excipient.

As used herein, the term “pharmaceutical compositions” or “pharmaceutical formulations” include tablets, pills, powders, liquids, suspensions, emulsions, granules, capsules, suppositories, or injection preparations.

The term “pharmaceutically acceptable excipients” selected from but not limited to binders, diluents, disintegrants, surfactants and lubricants. Suitable binders that can be include polyvinylpyrolidone, copovidone, starches such as pregelatinized starch, cellulose derivatives such as hydroxypropylmethyl cellulose, ethylcellulose, hydroxypropylcellulose and carboxymethylcellulose, gelatine, acacia, agar, alginic acid, carbomer, chitosan, dextrates, cyclodextrin, dextrin, glycerol dibehenate, guargum, hypromellose, maltodextrin, poloxamer, polycarbophil, polydextrose, polyethylene oxide, polymethacrylates, sodium alginate, sucrose, mixtures thereof; suitable diluents that can be include anhydrous lactose, lactose monohydrate, modified lactose, dibasic calcium phosphate, tribasic calcium phosphate, microcrystalline cellulose, silicified microcrystalline cellulose, powdered cellulose, maize starch, pregelatinized starch, calcium carbonate, sucrose, glucose, dextrates, dextrins, dextrose, fructose, lactitol, mannitol, sorbitol starch, calcium lactate or mixtures thereof; suitable disintegrants that can be include magnesium aluminometa silicate (or magnesium aluminum silicate), starch, pregelatinized starch, sodium starch glycolate, crospovidone, croscarmellose sodium, low-substituted hydroxypropyl cellulose, alginic acid, carboxy methyl cellulose sodium, sodium alginate, calcium alginate and chitosan; suitable lubricants that can be include (but are not limited to) magnesium stearate, stearic acid, palmitic acid, talc, and aerosil. Suitable surfactants that can be include (but are not limited to) polysorbate 80, polyoxyethylene sorbitan, polyoxyethylene-polyoxy-propylene copolymer and sodium lauryl sulphate; beta-cyclodextrin include (but are not limited to) sulfobutylalkyl ether-beta-cyclodextrin, betadex-sulfobutylether sodium, or hydroxypropyl-beta-cyclodextrin.

Pure amorphous form of Ubrogepant of formula-1 obtained according to the present invention and one or more pharmaceutically acceptable carriers for a calcitonin gene-related peptide receptor antagonist indicated for the acute treatment of migraine with or without aura in adults.

(3S,5S,6R)-3-amino-6-methyl-5-phenyl-1-(2,2,2-trifluoroethyl) piperidin-2-one 4-nitrobenzoate, (6S)-2′-oxo-1′,2′,5,7-tetrahydrospiro [cyclopenta [b]pyridine-6,3′-pyrrolo[2,3-b]pyridine]-3-carboxylic acid and Ubrogepant used in the preparation of amorphous form of Ubrogepant can be prepared according to process described in U.S. Pat. No. 8,754,096B2 and U.S. Pat. No. 9,174,989B2 or any other literature.

### PXRD Method of Analysis:

The PXRD analysis of compound of the present invention was carried out by using BRUKER-Axis/D8 ADVANCE (DAVINCI) X-Ray diffractometer using CuKα radiation of wavelength 1.5406 A°.

In view of the above description and the examples below, one of ordinary skill in the art will be able to practice the invention as claimed without undue experimentation. The foregoing will be better understood with reference to the following examples that detail certain procedures for the preparation of molecules, compositions and Formulations according to the present invention. All references made to these examples are for the purposes of illustration. The following examples should not be considered exhaustive, but merely illustrative of only a few of the many aspects and embodiments contemplated by the present disclosure.

### EXAMPLES

### Example-1: Preparation of Amorphous Form of Ubrogepant

Converted (3S,5S,6R)-3-amino-6-methyl-5-phenyl-1-(2,2,2-trifluoroethyl) piperidin-2-one 4-nitrobenzoate (38.28 g) into its free base using aqueous potassium phosphate solution. The obtained free base was dissolved in acetonitrile (150 ml). Aqueous sodium hydroxide solution was added to the mixture of acetonitrile (150 ml), water (150 ml) and (6S)-2′-oxo-1′,2′,5,7-tetrahydrospiro[cyclopenta[b]pyridine-6,3′-pyrrolo[2,3-b]pyridine]-3-carboxylic acid (25.0 g) at 25-30° C. and stirred at the same temperature. The above obtained (3S,5S,6R)-3-amino-6-methyl-5-phenyl-1-(2,2,2-trifluoroethyl) piperidin-2-one free base in acetonitrile was added to the reaction mixture at 25-30° C. and stirred at the same temperature. To this reaction mixture hydroxybenzotriazole hydrate (13.4 g) and 1-(3-dimethylaminopropyl)-3-ethyl carbodiimide hydrochloride (23.28 g) were added at 25-30° C. and stirred at the same temperature. Isopropyl acetate was added to the reaction mixture, separated the both organic and aqueous layer. The aqueous layer was extracted with isopropyl acetate. Combined the organic layers, washed with aqueous sodium bicarbonate solution, with aqueous citric acid solution and followed by with aqueous sodium bicarbonate solution. Distilled off the solvent completely from the organic layer under reduced pressure. Ethyl acetate was added to the obtained compound at 25-30° C., heated the mixture to 45-50° C. and stirred at the same temperature. Cooled the mixture to 25-30° C., petroleum ether was added and stirred at the same temperature. Filtered the precipitated solid, washed with petroleum ether and dried to get the title compound.

Yield: 46.0 g.

### Example-2: Preparation of Amorphous Form of Ubrogepant

Ubrogepant (45 g) was dissolved in methanol (135 ml) at 40-45° C. Filtered the mixture through hyflow bed and washed with methanol. Obtained filtrate was cooled to 25-30° C., water (450 ml) was added and stirred at the same temperature. Filtered the precipitated solid, washed with water and dried to get the title compound.

Yield: 42.0 g.

### Example-3: Preparation of Amorphous Form of Ubrogepant

Ubrogepant (5 g) was dissolved in methanol (20 ml) at 25-30° C. and stirred at the same temperature. Water (50 ml) was added to mixture at 25-30° C. and stirred at the same temperature. Filtered the precipitated solid, washed with water and dried to get the title compound. Yield: 4.2 g.

### Example-4: Preparation of Amorphous Form of Ubrogepant

Mixture of Ubrogepant (55 g) and methanol (165 ml) heated to 35-45° C. and stirred at the same temperature. Filtered the solution through hyflow bed for particle free and washed with methanol. Water (55 ml) was added to mixture at 25-30° C. and stirred at the same temperature. Filtered the precipitated solid, washed with water and dried to get the title compound.

Yield: 50 g. Residual Solvent Content: Methanol: 232 ppm, Ethanol: Not detected {ND}, Acetonitrile: ND, Ethyl acetate: 83 ppm, Pet ether: 76 ppm; Purity by HPLC: 99.75%; PXRD pattern of obtained compound is depicted in FIG. 1.

### Example-5: Preparation of Amorphous Form of Ubrogepant

Mixture of Ubrogepant (190 g) and methanol (570 ml) heated to 35-45° C. and stirred at the same temperature. Filtered the solution through hyflow bed for particle free and washed with methanol. The obtained filtrate was added to pre-cooled water (55 ml) at −5 to 5° C. and stirred at the same temperature. Filtered the precipitated solid, washed with water and dried to get the title compound.

Yield: 165 g.

We claim:

1. A pure amorphous form of Ubrogepant of formula-1

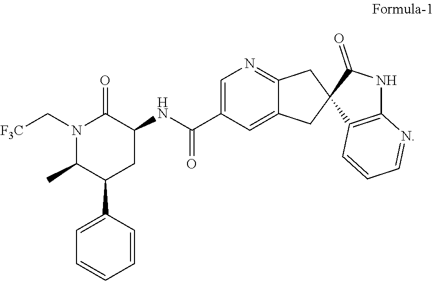 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F]

2. The pure amorphous form of Ubrogepant as claimed in claim 1, which is having a purity of greater than about 99% by HPLC.

3. The pure amorphous form of Ubrogepant as claimed in claim 1, which is essentially free of residual solvents.

4. The pure amorphous form of Ubrogepant as claimed in claim 1, which contains less than about 5% of crystalline forms of Ubrogepant.

5. The pure amorphous form of Ubrogepant as claimed in claim 1, which is stable.

6. The pure amorphous Ubrogepant as claimed in claim 5, which does not convert to any other solid forms when stored at a temperature of up to about 40° C. and at a relative humidity of about 25% to about 75% for about six months or more.

7. A process for the preparation of pure amorphous form of Ubrogepant of formula-1, the process comprising the steps of:
a) dissolving Ubrogepant of formula-1 in a solvent,
b) isolating amorphous form of Ubrogepant by combining with an anti-solvent.

8. The process as claimed in claim 7, wherein dissolving Ubrogepant in step-a) is carried out at a temperature ranging from about 25° C. to reflux temperature of the solvent used.

9. The process as claimed in claim 7, wherein the solvent in step-a) is selected from ester solvent, alcohol solvent, ketone solvent, nitrile solvent, polar-aprotic solvents or mixtures thereof.

10. The process as claimed in claim 7, wherein the anti-solvent is selected from ether solvents, hydrocarbon solvents, water or mixture thereof.

11. A pharmaceutical composition comprising amorphous form of Ubrogepant of claim 1 or claim 7, and a pharmaceutically acceptable carrier for use in the treatment of migraine.
